(19) United States
(12) Patent Application Publication
(10) Pub. No.: US 2023/0012734 A1
(43) Pub. Date: Jan. 19, 2023

(54) A WEARABLE DEVICE AND ASSOCIATED METHODS AND SYSTEMS

(71) Applicant: OPUM TECHNOLOGIES LIMITED, Grafton, Auckland (NZ)

(72) Inventor: Andrew John MCDAID, New Lynn, Auckland (NZ)

(73) Assignee: OPUM TECHNOLOGIES LIMITED, Grafton, Auckland (NZ)

(21) Appl. No.: 17/782,507

(22) PCT Filed: Dec. 4, 2020
(86) PCT No.: PCT/NZ2020/050166

§ 371 Date: Jun. 3, 2022

(30) Foreign Application Priority Data
Dec. 5, 2019 (NZ) 759887

(51) Int. Cl. A61B 5/00; A61B 5/11

(52) U.S. Cl. CPC A61B 5/6812; A61B 5/1121; A61B 5/7275; A61B 5/746; A61B 5/4585

(57) ABSTRACT

Systems and methods for monitoring acute:chronic workload ratio (ACWR) are described. An indicator of workload is received from a device worn or carried by a user. An acute workload is determined for the user based on the received indicator of workload over a first period of time. A chronic workload is determined for the user based on the received indicator of workload over a second period of time, where the first period of time is shorter than the second period of time. A current ACWR is determined for the user based on the acute workload and the chronic workload.

### 1 PRIORITY

This application is based on the provisional specification filed in relation to New Zealand Patent Application No. 759887, the entire contents of which are incorporated herein by reference.

### 2 FIELD OF TECHNOLOGY

The present technology is directed to wearable devices and systems in which they operate, and methods for aligning same and monitoring characteristics of a user wearing same.

### 3 BACKGROUND TO THE TECHNOLOGY

Wearable devices may be used in the rehabilitation, support, assistance, monitoring, diagnosis or prevention of afflictions and/or injuries of a human or animal wearer, whether neuromuscular, skeletal and/or otherwise. Such devices may be useful in the collection of data in relation to same.

The correct alignment of such devices may be important for a variety of reasons. For example, where the device is intended to provide a therapeutic effect—e.g. an osteoarthritis unloader brace used to offload weight from joint, or a range-of-motion brace to restrict the range-of motion of a joint — alignment can assist with achieving a correct or improved therapeutic effect. For devices intended to provide force or assistance to a user's joint, such as an active or passive exoskeleton, alignment can assist in achieving the proper effect. Further, where such devices are instrumented (i.e. configured to collect data regarding the wearer), alignment can be required to improve accuracy of the sensors. For example, where the device is intended to measure joint angle, misalignment may otherwise result in incorrect readings being obtained.

It is known to monitor workload in order to assess acute:chronic workload ratio (ACWR) for managing time/rate to return to activity such as sport or work or general activity, and to help more generally with injury prevention, recovery, fitness, or rehabilitation purposes. However, these measures are often subjective. Further, such measures need to be manually digitalised for logging and tracking—increasing the potential for errors in data entry, and/or failure to enter the data at all. Alternatively, where devices are used to monitor workload these may be generic in nature (for example, a pedometer or GPS tracker) and less reflective of workload on specific body parts of the user that are afflicted by a specific indication such as a knee ligament injury, or ankle sprain. Current methods also do not allow for automated and dynamic updates to ACWR thresholds to personalise and guide the user through recovery. Further, current methods of monitoring workloads rely on periodic recordal and analysis of workload, with ACWR is unable to be managed in real-time.

It may also be beneficial to objectively assess biomechanical characteristics of a person such as valgus angle or joint laxity. These measures are currently done in a clinical setting that is artificial and not representative of what happens throughout the day in the patients' real-world setting. It is also therefore not possible to track the cumulative effects or workloads of these biomechanical characteristics. These characteristics are also commonly subjective measures due to the setup and measurement time and complexity of existing objective measurement tools.

It is an object of aspects of the technology to provide a system, device and/or method for one or more of these purposes, or to overcome or ameliorate problems with existing systems, devices and methods. Alternatively, it is an object to provide an improved system, device and/or method for these one or more of these purposes. Alternatively, it is an object to at least provide the public with a useful choice.

### 4 SUMMARY OF THE TECHNOLOGY

It is an object of aspects of the technology to provide a system, device and/or method for one or more of these purposes Aspects of the present technology relate to a wearable device intended to be attached to at least one body part of a wearer. In examples the wearable device may be an orthosis, a prosthetic (i.e. an artificial body part such as an artificial limb), an exoskeleton, or a device primarily intended to monitor one or more characteristics of a wearer. Reference herein to an orthosis should be generally understood to means a support, brace, or splint used to support, align, or correct the function of movable parts of the body. For ease of understanding, examples of the present technology may be described herein in the context of an orthosis—however for completeness it should be understood that the features and principles disclosed are expressly contemplated as also having application to other devices, such as those noted above.

In examples, the wearable device may include a first body mounting portion and a second body mounting portion. In examples, the body mounting portions may be pivotally coupled relative to each other—for example directly coupled, or indirectly coupled via an intermediary component. For example, the wearable device may be a knee brace having an upper strut configured to be mounted upwardly of a knee of a wearer and a lower strut configure to be mounted downwardly of the knee of the wearer, each strut coupled to an intermediary pivot assembly.

In examples, the wearable device may include one or more sensors. It should be appreciated that a variety of sensors may be used, but generally the sensor may be configured to measure an indicator of a physiological variable of the user—for example biomechanical parameters, especially kinetic and/or kinematic parameters of the patient, or variables from which these may be derived. By way of example, the one or more sensors may include one or more of: motion and/or orientation sensors (for example one or more of accelerometers, magnetometers, and gyroscopes), including integrated devices such as an inertial measuring unit (IMU); angular displacement sensors (for example incremental sensors such as rotary encoders, or absolute position sensors), including in combination to measure angular motion in multiple directions (for example, an instrumented linkage mechanism); electrogoniometers; force sensors (for example, load cells, and pressure sensors in foot insoles); and physiological sensors, for example, a electromyography (EMG) sensor, a thermometer, a heart rate sensor, a blood pressure sensor, a blood oxygen level sensor, etc.

In examples, processing and/or recording of sensor data may be performed by one or more of at least: dedicated processor(s) of the wearable device, processor(s) of a device including a reference sensor, processor(s) of a user device (for example, a personal computing device such as a smart phone, tablet, or personal computer), and remote processing means (for example, a server or cloud computing services). The various components described herein may communicate using any suitable means known to those skilled in the art of data communication, including wired and wireless communication protocols. In exemplary embodiments, data from the wearable device and/or reference sensor may be stored locally (e.g. on removable memory device) and transferred by physical removal of the storage device. Display of a determination of symmetry, and in exemplary embodiments the underlying data, may be displayed on any suitable display device.

According to one aspect of the present technology there is provided a method of aligning a wearable device with at least one body part of a wearer. According to another aspect of the present technology the wearable device may be configured to assist with performing alignment.

In examples, real-time feedback of the orientation of at least one component of the wearable device is provided in order to enable determination of orientation relative to a reference coordinate system. Alignment is important to provide accurate monitoring of joint or body position or orientation that can then be used as inputs during processing of sensor outputs, e.g. to determine activity or posture. Alignment is also important to provide improved therapeutic effect and force/torque interaction with the wearer of the device (for example, accurate range -of-motion restriction, or force/support from an exoskeleton). Due to the varying shape and size of wearers' limbs, as well as soft tissue deformation and wearers' clothing, it can be difficult to align a wearable device with the limb. In addition, sensors such as accelerometers or IMUs rely on wold coordinate systems (e.g. gravity and bearing) as the reference, so it is important the fitter knows the alignment of the wearable device relative to these coordinate systems, which may not be immediately apparent without specific feedback.

In examples, the real-time feedback is provided by a visual indication of orientation. In an example, at least one spirit level may be provided on one or more components of the wearable device—for example one or more of the struts, and/or intermediary pivot assembly. In an example, the spirit level may be removably attached to another component of the wearable device—for example using one or more fastening means (e.g. magnetic, snap fasteners, hook and loop material). In alternative examples, the at least one spirit level may be permanently attached to the wearable device. In an example the orientation of the spirit level may be selectively adjustable—for example rotatable between a vertical orientation and a horizontal orientation.

In use, the spirit level may be used to align the wearable device when the body part(s) of the wearer is in a known orientation. For example, the spirit level may be used to determine when the wearable device is vertical when attaching to the leg of a wearer while the wearer is standing comfortably/naturally upright. In another example, the spirit level may be used to determine when the wearable device is horizontal when attaching to the leg of a wearer while the wearer is lying flat, or sitting with their leg extended.

In examples the visual indication of orientation may be static in form, for example a line or arrow. The visual indication may contrast with the component of the wearable device to which it is provided—for example using colour, texture, recessing, or elevation. In examples, the location of the visual indication may be adjustable. For example, the location of the visual indication may be based on the circumference of the wearer's leg, with the location of the visual indication aligned with a readily observable aspect of the wearer's leg (e.g. on the sagittal plane in a forward walking direction). This position may be established, for example by a trained professional in clinic, and then remain in place to be used by the wearer each time the wearable device is put on, and/or as the wearable device is worn throughout the day to improve the likelihood of misalignment being detected. Circumferential distance can be set for different size users, or as the leg swells/shrinks, to ensure that indicator is always in front of the leg. In an example the visual indication may be provided on a component having another purpose—for example a strap around the leg of the wearer. In an alternative example the visual indication may be provided on a dedicated member extending around at least a portion of the wearer's leg from another component of the wearable device.

In examples, at least one compass may be provided on one or more components of the wearable device. The compass may be used to determine the orientation of the wearable device relative to one or more geographic cardinal directions (i.e. bearing). Bearing may be important when the alignment is happening in the horizontal plane, e.g. if fitting a shoulder orthosis, if the wearer's body/torso if facing north, and the arm is facing east, then that shoulder angle is at zero degrees with the torso. When fitting an orthosis the user maybe asked to hold their shoulder at 90 degrees (i.e. directly out in front of their body) and then the orthosis can give feedback to see when this also reads 90 degrees and be adjusted accordingly.

In examples, the wearable device may include at least one orientation sensor. In examples the orientation sensor may include one or more of: an accelerometer, a magnetometer, and a gyroscope. In an example, the orientation sensor may include an inertial measurement unit (IMU).

In examples, at least one orientation indicator may be provided to output a signal indicative of the wearable device, or component thereof, being in a particular orientation. In an example the orientation indicator may be provided at the wearable device—for example, one or more of: visual indicators (e.g. a light), audible indicators (e.g. a buzzer or beeper), and/or haptic indicators (e.g. a vibrating device). In examples the orientation indicator may be controlled to indicate when the orientation is achieved, and/or when the orientation is not achieved. In examples, the orientation indicator may be controlled to output a first signal when the orientation is achieved (for example, a green light), and output a second signal when the orientation is not achieved (for example, a red light).

According to an aspect of the present technology there is provided a calibration method for one or more sensors associated with a wearable device. In examples in which the wearable device has one or more sensors, it may be desirable to calibrate those sensors in order to relate the sensor output to one or more of the wearer's body parts, for error compensation, and/or to maintain accuracy (e.g. to compensate for sensor drift). In the example of a knee orthosis, sensors may be provided for measuring angles between the body mounting portions. It may be desirable to calibrate the sensors using a reference point, such as the wearer standing straight (during which times the angles should be zeroed). Such calibration may be performed before, after, and/or during a monitored activity.

In examples, calibration may be performed on receiving a user input while in a predetermined position to provide a reference. For example, the user input may be provided by selection of a physical button on the wearable device, or a virtual selectable element on a user interface located remotely from the wearable device (for example, a GUI of an application operating on a computing device), a voice command, or any other suitable means.

In examples, calibration may be performed automatically on detection of a predetermined event or activity. For example, the wearer's posture during activity may be monitored using one or more motion or orientation sensors, and a standing upright posture may be identified within this activity. On detecting the standing upright posture, the sensors may be calibrated accordingly.

According to an aspect of the present technology there is provided a wearable device configured to measure at least one characteristic of a joint, or a body part associated with a joint, of a wearer.

According to an aspect of the present technology there is provided a wearable device configured to measure angulation of a bone or joint of a wearer. For example, the wearable device may be configured to measure varus deformity, or valgus deformity.

In an example, the wearable device may include a first body mounting portion and a second body mounting portion, wherein at least a portion of at least one of the body mounting portions is flexible. The wearable device may further include at least one sensor associated with the flexible portion of the body mounting portion, configured to output a signal indicative of angulation of a body part to which the wearable device is mounted. For example, the at least one sensor may be a flex sensor, a bend sensor or a stretch sensor (where stretch is used as an indirect measure of bending, for example a strain gauge).

In an example, the wearable device may include a first body mounting portion and a second body mounting portion, wherein the wearable device may be configured such that the at least one of the body mounting portions may pivot in the frontal plane. The wearable device may further include at least one angle sensor, configured to output a signal indicative of angulation of a body part to which the wearable device is mounted.

In examples, the sensor output may be monitored during an activity such as walking, or other activities such as specific rehabilitation exercises or assessments.

According to an aspect of the present technology there is provided a wearable device configured to measure at least one indicator of joint laxity of a joint of a wearer. Joint laxity, or loose joints, occurs where two body segments either side of a joint are not rigidly coupled as in normal joints, often caused by loose ligaments of the joint (for example in the feet, knee, elbow or other joints). Joint laxity can cause decreased co-ordination, weakness, and result in increased probability of injury.

In examples, the wearable device may include a first body mounting portion and a second body mounting portion, and a device joint between the first body mounting portion and the second body mounting portion. In examples, the device joint may be configured to provide three planar degrees of freedom. In examples, the device joint may be locked in rotation (e.g. through use of mechanical limitations, such as pins in a rotary joint). For example, where the wearable device is to be mounted across a knee of the wearer, in one embodiment valgus rotation may be restricted (i.e. only sagittal plane motion possible), and the rotation locked through a pin mechanism so only relative planar movement of the knee can be sensed i.e. not knee rotation. In examples, the device may be configured to have planar joint motion—i.e. restricting movement in the joint to planar joint motion. For example, the device may include one or more sliding mechanisms which restrict movement to planar movement only.

In examples, at least one sensor may be provided to sense relative movement between the first body mounting portion and the second body mounting portion. In examples, the sensor may be a hall effect magnetic sensor, or an optical sensor.

In an example, the device joint may include at least three linear joints having a three-axis orthogonal arrangement, each linear joint having an associated sensor configured to output an indication of movement in the associated linear joint.

According to an aspect of the present technology there is provided a system configured to measure at least one indicator of patella motion of a wearer. In examples the system may include one or more motion parameter sensors, such as inertial measurement units (IMU), or combination of individual sensors such as accelerometers, gyroscopes and magnetometers.

In an example, a first sensor, or combination of sensors, may be provided on the thigh or shank of the wearer, and a second sensor, or combination of sensors, may be provided on the corresponding patella of the wearer. Motion of the patella relative to the wearer's thigh or shank may be determined during manipulation of the patella by the wearer or a clinician, for example for assessment of exercise.

In another example, a first sensor, or combination of sensors, may be provided on the patella of the wearer, and the corresponding thigh or shank of the wearer assumed to be stationary during manipulation of the patella in order to determine relative movement of the patella. In examples a restraining mechanism may be provided to ensure the wearer's thigh or shank remains stationary during manipulation, such as a moulded jig or strap system to restrain the limb.

According to one aspect of the present technology there is provided a method of measuring workload using a wearable device. According to one aspect of the present technology there is provided a wearable device configured to measure an indicator of workload of a wearer. According to one aspect of the present technology there is provided a method of determining acute:chronic workload ratio (ACWR) based on an indicator of workload measured by a wearable device worn by the wearer.

It is envisaged that ACWR may be used as a metric for interpreting how quickly or aggressively a person is progressing with their rehabilitation. Accordingly, in examples the determined ACWR may be used as a metric for assessing a level of risk to a person progressing or pushing themselves too far, or alternatively may be used to provide guidance as to progression of rehabilitation (for example, providing recommendations regarding a level of activity within a predetermined time period).

In examples, the wearable device includes at least one sensor configured to measure the indicator of workload. By way of example, the sensor may be configured to measure the angle of rotation of a joint to which the wearable device is mounted. In such an example, it is envisaged that workload may be determined as accumulated angle over time. As another example, the sensor may be configured to measure distance travelled by the wearer. As another example, the senor maybe configured to measure activity or posture by the wearer, such as standing, sitting, walking. As another example the sensor may be used in conjunction with biomechanical modelling to estimate joint load, e.g. joint load, and/or time patient is weight bearing. These measures of workload are more targeted for specific indications such as knee ligament injuries, arthritis or surgical interventions and can give unique insight into the state of the wearer throughout the day, not just when in physical activity such as walking. For example, a user may go through large number of knee flexion/extension exercises while sitting on a chair to try and increase range-of-motion, but this cannot be measured by GPS or pedometer to manage the patient's knee ACWR. As a further example there may be an interest to manage the ACWR for a specific activity like time standing, or knee elevated after a surgery.

In examples the wearable device may be a user device such as a smart watch or smart phone, having one or more sensors capable of capturing motion parameters indicative of activity such as step count, stride length, double stance time, and/or others.

Typically when ACWR is used in sports for athlete training or returning from injury the ACWR for their training is of interest. However, for recovery from injury the wearer may not be an athlete or even very active, so measures of workload are needed not just while in specific training sessions but throughout the day in activities of daily living. Integration into a brace or other wearable device (such as a smart watch or smart phone worn or otherwise carried by the wearer) which is worn throughout recovery also allows the workload to be calculated longitudinally (i.e. over long periods of time such as months). These workload measures can then be used to manage the recovery and return to sport, work, and/or activity in a holistic manner.

In examples, workload may be determined over one or more predetermined periods of time. In examples, acute workload may be determined as workload over a first period of time, and chronic workload may be determined as workload over a second period of time, where the first period of time is shorter than the second period of time. For example, the first period of time (i.e. for acute workload) may be a day, and the second period of time (i.e. for chronic workload) may be a week.

In examples, chronic workload may be determined as an average value of workload over the second period of time. In examples, the average may be determined as a moving average.

In examples, a current value for ACWR may be used to determine a current risk level to the user. In examples, a current value for ACWR may be compared against one or more predetermined thresholds to determine the current risk level, more particularly on an escalating scale. For example, a first threshold may be associated with a cautionary risk level, a second threshold may be associated with a warning risk level, and a third threshold may be associated with an alert risk level. The level of risk may be associated with consequences, for example at a higher level of ACWR the probability of re-injury is higher than at lower level of ACWR. As a further example, ACWR below a first threshold may indicate under training or rehabilitation, between the first threshold and a second threshold may indicate optimal recovery, and above the second threshold may indicate a danger zone. The provision of such regions may assist with improving the ease of understanding by the wearer.

In an example, the first threshold may be about 1, the second threshold may be about 1.5, and the third threshold may be about 2. It should be appreciated that these values are provided by way of example, and are not intended to be limiting to all embodiments.

In examples, the one or more thresholds may be dynamic—i.e. may be adjusted in response to one or more parameters. For example, the one or more thresholds may increase based on time since an event (e.g. an injury, or surgery) In another example, the one or more thresholds may be adjusted based on performance metrics—for example raw testing scores for one or more activities, or rate of improvement over time—where below expectation metrics may reduce the thresholds, while above expectation scores may increase the thresholds. In another example, the thresholds may be based on demographics of the patient, such as age, height, weight, gender etc. In another example, thresholds might be based on the desired workload goal at a long distance in time (i.e. when fully recovered in nine months, should be doing one million degrees of motion per day) and then adjust the ACWR for the current day based on the trajectory to achieve that goal.

In examples, guidance for future rehabilitation activity of the user may be determined based on the current ACWR. In particular, the guidance may be based on a desired ACWR within a future period of time, for example a level of activity recommended for a predetermined time period in order to achieve rehabilitation goals while remaining below a predetermined ACWR.

In examples, a notification may be issued regarding a current acute:chronic workload ratio experienced by a user. In examples, the notification may be issued on reaching a predetermined threshold. In another example a predictive model may be used, which may also be based on trends from previous days of activities, that provides a warning at intermediary time points throughout the day, for example in the morning if the wearer has done a high percentage (e.g. 90%) of their allowable workload for that day, then a notification might be presented that they should rest for the remainder of the day to remain within the allowable ACWR for that day. Further, if that individual consistently does a high percentage of their workload before a particular time, but otherwise remains within allowable ACRW for the day, then this trend may be recognised and warnings not issued at that point.

In examples, the notification may be issued through a user interface accessed by a computing device (for example, an application operating on a smart phone, or a web application accessed through a browser), or delivered as an electronic message (for example, a SMS message or email). In examples, the notification may be issued at the wearable device—for example, a warning light, audible tone, or haptic feedback.

In examples, the notification may be issued to the wearer. In examples, the notification may be issued to another party, for example a clinician or trainer of the wearer.

Further aspects of the disclosure, which should be considered in all its novel aspects, will become apparent to those skilled in the art upon reading of the following description which provides at least one example of practical application of the disclosure.

### 5 BRIEF DESCRIPTION OF THE DRAWINGS

One or more embodiments of the disclosure will be described below by way of example only, and without intending to be limiting, with reference to the following drawings, in which:

FIG. 1 is a schematic diagram showing features of a wearable device system according to an aspect of the present technology;

FIG. 2-1 is a front perspective view of an exemplary wearable device in the form of a knee brace according to an aspect of the present technology;

FIG. 2-2 is a front perspective view of another exemplary wearable device in the form of a knee brace according to an aspect of the present technology;

FIG. 3 is a front view of an exemplary wearable device in the form of an elbow brace according to an aspect of the present technology;

FIG. 4-1 is a side view of an exemplary wearable device mounted to a standing wearer, configured to be aligned with a coordinate system according to an aspect of the present technology;

FIG. 4-2 is a side view of the exemplary wearable device mounted to a seated wearer, configured to be aligned with a coordinate system according to an aspect of the present technology;

FIG. 4-3 is a front perspective view of an exemplary wearable device to be mounted to a wearer, having a visual indicator of alignment according to an aspect of the present technology;

FIG. 4-4 is a top view of an exemplary wearable device, having a visual indicator of alignment according to an aspect of the present technology;

FIGS. 5-1 to 5-3 are front views of an exemplary wearable device mounted to the leg of a wearer for measuring angulation of the leg according to an aspect of the present technology;

FIG. 6-1 is a side view of an exemplary wearable device mounted to a leg of a wearer, for measuring joint laxity according to an aspect of the present technology;

FIG. 6-2 is a side view of an exemplary joint mechanism for a wearable device according to an aspect of the present technology;

FIG. 7 illustrates a sensor arrangement for sensing characteristics of patella movement according to an aspect of the present technology;

FIG. 8 is a flow diagram of an exemplary method of monitoring workload of a wearer of a wearable device according to aspects of the present technology.

### 6 DESCRIPTION OF EXAMPLES OF THE TECHNOLOGY

It will be understood that the particular examples described herein are not intended to be limiting to all embodiments of the present technology. The various examples may share one or more common characteristics and/or features. It should be appreciated that one or more features of any one example may be combinable with one or more features of one or more other examples.

6.1 Exemplary Wearable Device System

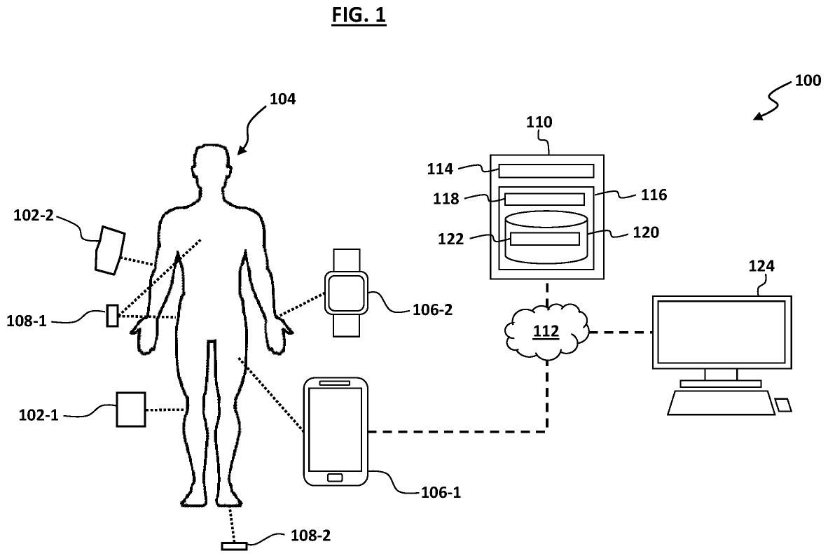

FIG. 1 is a schematic illustration showing features of a system 100 according to certain embodiments of the technology. The system 100 includes one or more wearable devices 102 (for example, knee orthosis 102-1 and/or elbow orthosis 102-2) configured to be mounted to a corresponding body part (s) of a body of a patient 104 in use. In examples, the system 100 further includes one or more reference sensors, including intelligent user devices 106 (for example, smart phone 106-1 and/or smart watch 106-2) and/or dedicated reference sensor devices 108 (for example, an inertial measurement unit (IMU) 108-1 and/or smart insole 108-2).

In exemplary embodiments, data from one or more of the wearable devices 102, user devices 106, and/or reference sensor device 108 may be communicated to a remote processing service 110 via a network 112 (for example a cellular network, or another network potentially comprising various configurations and protocols including the Internet, intranets, virtual private networks, wide area networks, local networks, private networks using communication protocols proprietary to one or more companies—whether wired or wireless, or a combination thereof). For example, the smart phone 106-1 may operate an application capable of interfacing with the data management service 110.

Among other functions, the remote processing service 110 may record data, perform analysis on the received data, and report to one or more user devices. In this exemplary embodiment, the remote processing service 110 is illustrated as being implemented in a server—for example one or more dedicated server devices, or a cloud based server architecture. By way of example, cloud servers implementing the remote processing service 110 may have processing facilities represented by processors 114, memory 116, and other components typically present in such computing environments. In the exemplary embodiment illustrated the memory 116 stores information accessible by processors 114, the information including instructions 118 that may be executed by the processors 114 and data 120 that may be retrieved, manipulated or stored by the processors 114. The memory 116 may be of any suitable means known in the art, capable of storing information in a manner accessible by the processors, including a computer-readable medium, or other medium that stores data that may be read with the aid of an electronic device. The processors 114 may be any suitable device known to a person skilled in the art. Although the processors 114 and memory 116 are illustrated as being within a single unit, it should be appreciated that this is not intended to be limiting, and that the functionality of each as herein described may be performed by multiple processors and memories, that may or may not be remote from each other.

The instructions 118 may include any set of instructions suitable for execution by the processors 114. For example, the instructions 118 may be stored as computer code on the computer-readable medium. The instructions may be stored in any suitable computer language or format. Data 120 may be retrieved, stored or modified by processors 114 in accordance with the instructions 118. The data 120 may also be formatted in any suitable computer readable format. Again, while the data is illustrated as being contained at a single location, it should be appreciated that this is not intended to be limiting—the data may be stored in multiple memories or locations. The data 120 may include databases 122 storing data such as historical data associated with one or more of the one or more of the wearable devices 102, user devices 106, and/or reference sensor devices 108, and the results of analysis of same.

It should be appreciated that in exemplary embodiments the functionality of the remote processing service 110 may be realized in a local application (for example, on smart phone 106-1, or another personal computing device 124), or a combination of local and remote applications. Further, it should be appreciated that data may be transferred from one or more of the devices by other means—for example wired communication links, or transfer of storage devices such as memory cards.

The results of analysis, and/or underlying data, may be displayed on any suitable display device—for example smart phone 106-1, or computing device 124.

### 6.2 Knee Brace

FIG. 2-1 shows an exemplary wearable device in the form of an orthosis system particularly suited for mounting proximate a knee (not shown) of the patient (not shown)—herein referred to as first knee brace 200-1—as described in PCT application PCT/NZ2018/050085, the contents of which are incorporated herein by reference. In this embodiment, the knee brace 200-1 includes a body mounting portion having a first brace portion 202-1 and a second brace portion 202-2. In use, the first brace portion 202-1 is mounted upwardly of the knee of the patient and the second brace portion 202-2 is mounted downwardly of the knee of the patient. The first brace portion 202-1 and the second brace portion 202-2 are pivotably coupled via pivot assemblies 204-1 and 204-2. This makes the orthosis system 200-1 suited to use in bracing a pivoting joint of the body, such as the knee.

In other embodiments of the invention the first and second brace assemblies are moveably coupled in some other manner, for example through a sliding coupling. Such embodiments may be suitable for use in bracing an extendable part of the body, for example. In yet other embodiments, the brace of an orthosis system may be provided as a flexible sleeve, such as a continuous compression sleeve. A first portion of the sleeve is a first body mounting portion to be worn on one side of the wearer's joint, and a second portion of the sleeve coupled to (i.e. integrally formed with) the first portion is a second body mounting portion to be worn on an opposite side of the wearer's joint. In the embodiment illustrated, modules 206-1 and 206-2 are removably coupled to the pivot assemblies 204-1 and 204-2. One or both of the modules 206-1 and 206-2 may be configured as sensing modules. While the modules 206-1 and 206-2 are illustrated as being on the sides of the patient's knee, in other embodiments the orthosis system may be configured to mount modules in other positions in relation to the body.

FIG. 2-2 shows a second knee brace 200-2 includes a body mounting portion having a first brace portion 202-1 and a second brace portion 202-2. In use, the first brace portion 202-1 is mounted upwardly of the knee of the patient and the second brace portion 202-2 is mounted downwardly of the knee of the patient. In this exemplary embodiment, the brace portions 202 are stiff arms (for example made of aluminium) having strap mounting features (for example slots 208) for positioning flexible straps (not illustrated) for mounting the knee brace 200-2 to a wearer. In this embodiment, sensing module 206 is removably coupled to the pivot assembly 204 of the knee brace 200-2. The sensor module 206 includes a rotational knee movement sensor, and an IMU for sensing of thigh movement.

### 6.3 Elbow Brace

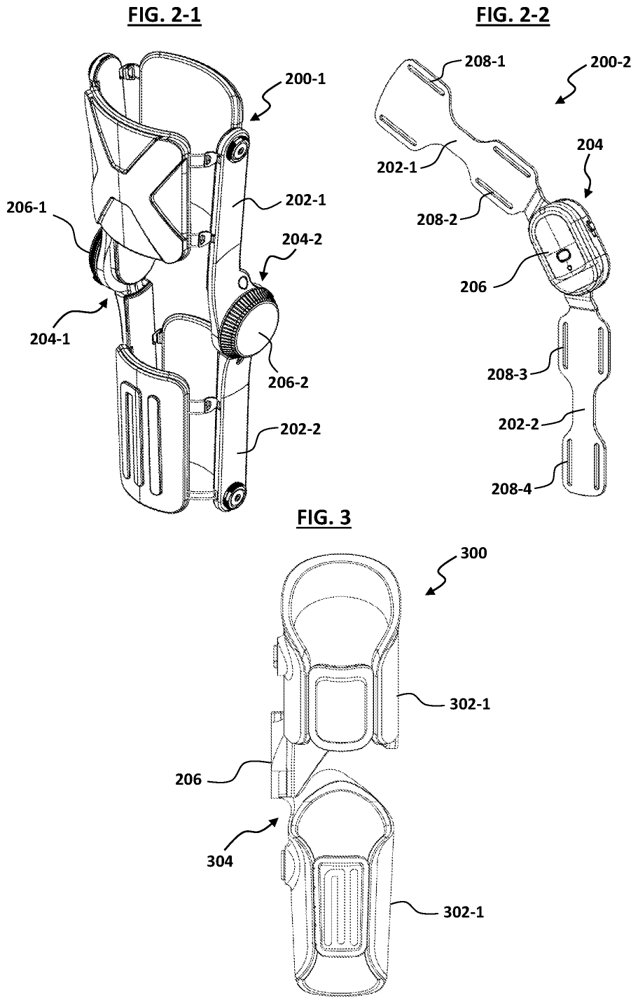

FIG. 3 shows an exemplary wearable device particularly suited for mounting proximate an elbow (not shown) of the patient (not shown)—referred to herein as elbow brace 300—as described in PCT application PCT/NZ2018/050085, the contents of which are incorporated herein by reference. It will be noted that the body mounting portion in this embodiment comprises a first brace portion 302-1 configured to wrap around a first portion of the patient's arm. Similarly, the second brace portion 302-2 is configured to wrap around another portion of the patient's arm. The first brace portion 302-1 and the second brace portion 302-2 are pivotably coupled via pivot assembly 304-1, to which a sensor module 206 is mounted.

### 6.4 Sensing Module

Exemplary embodiments of the sensing module 206 comprises sensor components configured to detect, record, process and/or transmit data relating to the movement and/or rotation of the orthosis system or components thereof. The sensor components may additionally or alternatively detect, record, process and/or transmit data pertaining to the patient's physical activity and/or physiology. This may include parameters such as joint kinematics (such as joint angle, joint velocity, joint torque, and/or joint acceleration), limb accelerations, limb rotations, limb and/or joint loads, muscle force, muscle strength, muscle velocity, electrical activity, temperature, pH, perspiration, heart rate, blood pressure and/or other bio-signals. Example sensors include rotary encoder, optical and magnetic sensors.

The sensing module 206 may comprise further components to enable the detection and recording of such data. For example, the sensing module 206 may comprise an accelerometer, gyroscope and/or magnetometers. The sensing module 206 may additionally or alternatively comprise physiological sensors, for example a thermometer, electromyography (EMG) sensor, heart rate sensor, blood pressure sensor, blood oxygen level sensor, etc.

Sensing module 206 may comprise a transmitter for transmitting data and/or signals obtained by or through the sensor components to a remote location, for example by RF, Bluetooth, Wi-Fi or any other remote communication protocol. Sensing module 206 may also comprise one or more processors configured to process the data/signals. The sensor components may further comprise a receiver configured to receive data/signals remotely from an external source, such as external control signals. Data may be stored or received by the sensing module 206 through a physical data storage device such as a memory card, USB stick or the like.

Other sensors may be provided, comprised in or separate from a sensing module 206. For example, the wearable device 200 may also comprise a torque sensing module comprising one or more sensors for monitoring joint interaction torque between the patient and the body mounting portion. For example, such a sensor(s) may monitor relative displacement between two or more components of the device 200, for example the first and second brace portions respectively, to enable a torque sensor to sense torque between the first and second brace portions. Torque sensing may be performed when the first and second brace portions are locked, or there is some resistance between them. It should therefore be appreciated that a torque sensing module may also incorporate a locking mechanism to substantially prevent movement (e.g. rotation) between the first and second brace portions, such as described in PCT application PCT/NZ2018/050085.

A person skilled in the art will understand that a number of sensor types may be suitable for measuring characteristics of a patient's biomechanics. For example, a rotary encoder may be used to measure an angle of displacement between the first and second brace portions. Alternatively or additionally an inertial measuring unit(s) (IMU) may be attached to one or each of the first and second brace portions to measure the angle of displacement. An angle of displacement may be used to infer a resistance to motion level, by calibration of a known resistance element with respect to the amount of relative movement between the brace assemblies, or conversely a resistance measurement such as torque or force may be used to infer angle. A strain gauge may be provided to a compliant/resilient element such as a spring or elastomeric block to measure force or torque, and/or a position of a spring element may be used to indicate a resistance to motion level.

### 6.5 Aligning Wearable Device

In certain forms of the present technology, real-time feedback of the orientation of at least one component of the wearable device is provided in order to enable determination of orientation relative to a reference coordinate system.

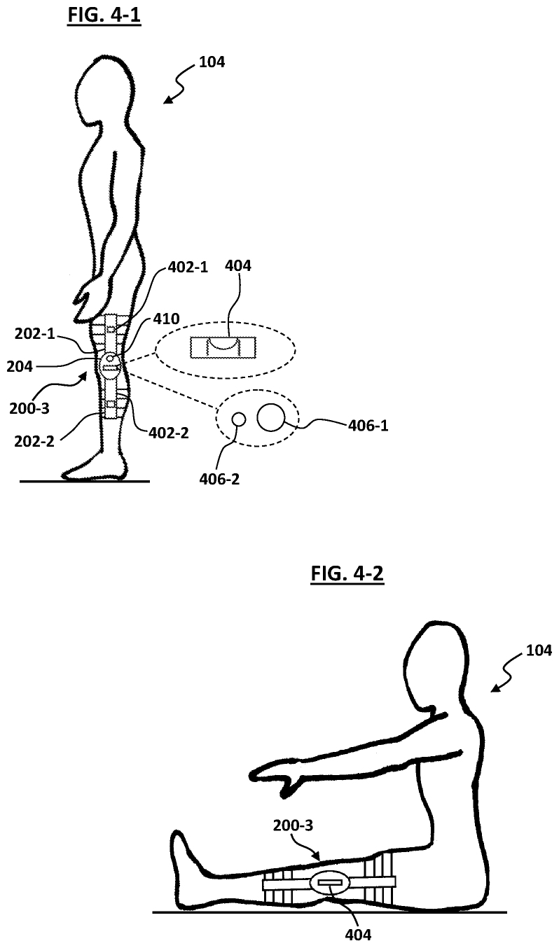

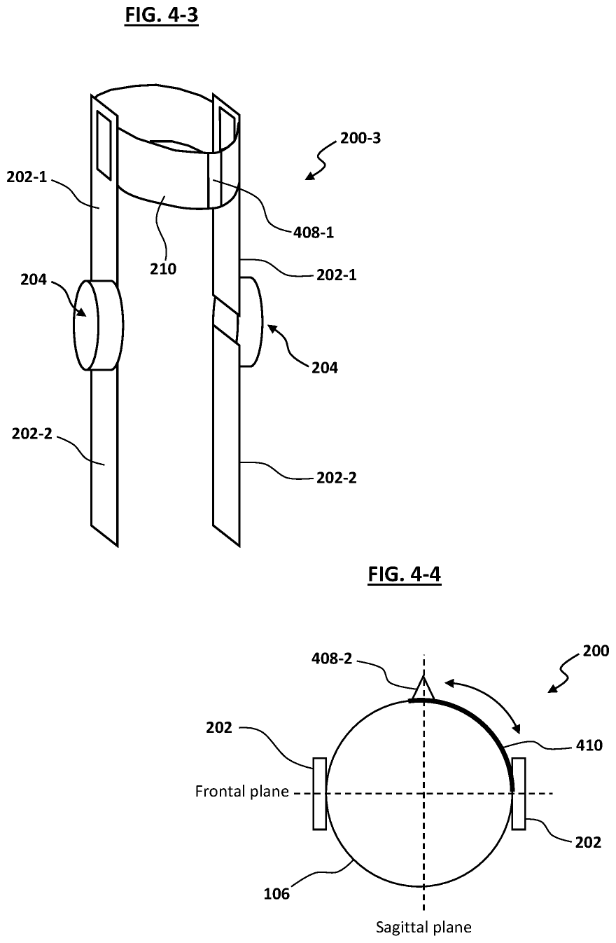

FIG. 4-1 shows an exemplary wearable device in the form of a third knee brace 200-3, mounted to the knee of wearer 104. The knee brace 200-3 is configured to provide a visual indication of orientation of the knee brace 200-3 to a reference coordinate system. In examples, the knee brace 200-3 may include visual indicators at a central location 400 (for example on a pivot assembly 204), an upper location 402-1 on upper brace portion 202-1, and/or a lower location 402-2 on lower brace portion 202-2.

In a first example, the visual indicator may be provided by a spirit level 404. In the example illustrated in FIG. 4-1, the spirit level 404 is intended to be horizontal when the knee brace is generally vertical in orientation (i.e. is configured to indicate vertical orientation). In order to align the knee brace 200-3, the wearer 104 may stand up straight to provide a reference, and the knee brace 200-3 moved to obtain a vertical orientation using the spirit level 404. In an alternative example utilising a spirit level, as illustrated in FIG. 4-2, the spirit level 404 is intended to be horizontal when the knee brace is generally horizontal in orientation (i.e. is configured to indicate horizontal orientation). In order to align the knee brace 200-3, the wearer 104 may sit or lie down on the ground or another level surface (such as on a bed or bench) such that their leg is fully extended to provide a reference, and the knee brace 200-3 moved to obtain a horizontal orientation using the spirit level 404.

In a second example, the knee brace 200-3 may include an orientation sensor, for example an IMU, configured to determine orientation of the knee brace 200-3. A visual indicator may be provided in the form of a light 406, illuminated to indicate the orientation of the knee brace 200-3. In the example illustrated, a first light 406-1 and a second light 406-2 are provided. The first light 406-1 may be illuminated when the knee brace 200-3 is vertical—i.e. straight up and down relative to the ground—while the second light 406-2 may be illuminated while the knee brace 200-3 is not vertical. In an alternative example, a single light such as a multi-colour LED may be used to indicate different states of orientation (e.g. a first colour indicating alignment with vertical, and a second colour indicating non-alignment).

In alternative examples, an audible indicator (for example, a buzzer) and/or a haptic feedback indicator (for example, a vibration generator) may be used to indicate alignment or non-alignment.

In a third example, referring to FIG. 4-3, a visual indicator (in this example a vertical line 408-1) may be provided on a strap 210 of the knee brace 200-3. The vertical line 408-1 may be releasably attached to the strap 210 (for example using hook and loop material, snap fasteners, clips, or any other suitable means) to allow for placement at the front of the wearer's leg on the sagittal plane in a forward walking direction. Releasable attachment allows for placement based on the circumference of the wearer's leg, such that the vertical line 408-1 is accurately positioned in the front of the leg. If the knee brace 200-3 becomes misaligned during use, for example by twisting about the wearer's leg, this may be detected through observation of the position of the vertical line 408-1.

In a further example shown in FIG. 4-4, a raised visual indicator 408-2 may be provided on a dedicated support member 410 extending around at least a portion of the wearer's leg 106 from another component of the wearable device (for example a brace portion 202). In examples, the visual indicator 408-2 may be releasably attached to the dedicated support member 410 in order to align its position relative to the sagittal plane in front of the wearer's leg. In alternate examples the dedicated support member 410 may be moveable relative to the wearable device, for example a ratcheting rigid strap, or sliding arm.

According to an aspect of the present technology there is provided a calibration method for one or more sensors associated with the wearable device. In examples in which the wearable device has one or more sensors, it may be desirable to calibrate those sensors in order to relate the sensor output to one or more of the wearer's body parts, for error compensation, and/or to maintain accuracy (e.g. to compensate for sensor drift).

In the example of a knee brace 200-3 as shown in FIG. 4-1, sensors may be provided for measuring angles between the body mounting portions 202-1 and 202-2. It may be desirable to calibrate the sensors using a reference point, such as the wearer standing straight (during which times the angles should be zeroed). Such calibration may be performed before, after, and/or during a monitored activity.

In certain forms of the present technology, calibration may be performed on receiving a user input while in a predetermined position to provide a reference. For example, the user input may be provided by selection of a physical button 410 on the knee brace 200-3. In another example, the user input may be received on selection of a virtual selectable element on a graphical user interface displayed on smart phone 106-1.

In examples, calibration may be performed automatically on detection of a predetermined event or activity. For example, the posture of the wearer 104 during activity may be monitored using one or more motion or orientation sensors, and a standing upright posture may be identified within this activity. On detecting the standing upright posture, the sensors may be calibrated accordingly (for example, providing a zero reference for angle sensors of knee brace 200-3. As a further example, the system may provide a message to the wearer asking them to confirm if they are standing upright, and if the user indicates that they are, then the system calibrates.

### 6.6 Joint Characteristic Measurement

In certain forms of the present technology, a wearable device is configured to measure at least one characteristic of a joint, or a body part associated with a joint, of a wearer.

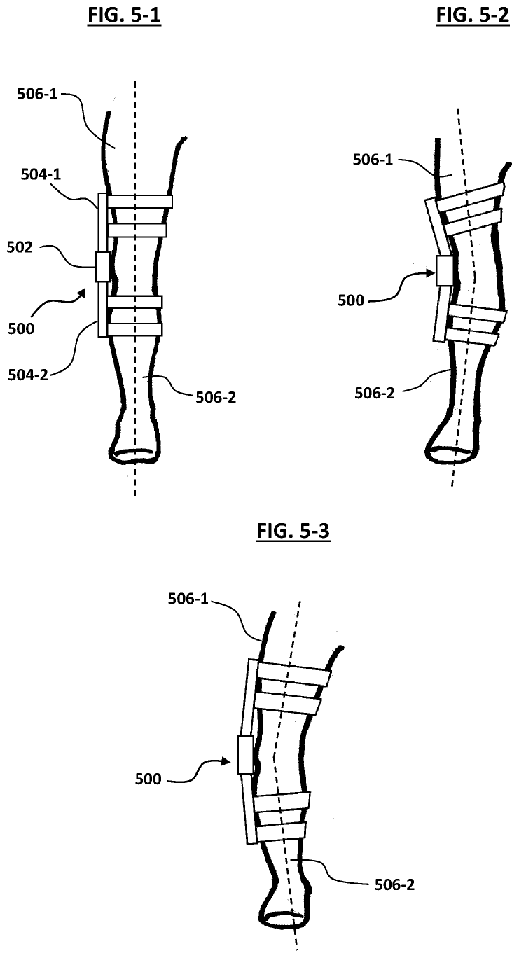

In a first example, a wearable device configured to measure angulation of a bone or joint of a wearer. For example, the wearable device may be configured to measure varus deformity, or valgus deformity. Referring to FIG. 5-1, a knee brace 500 includes a central portion 502, a first body mounting portion in the form of upper strut 504-1, and a second body mounting portion in the form of lower strut 504-2. The upper strut 504-1 and the lower strut 504-2 are made of a flexible material which retains sufficient rigidity to hold its form in the absence of loading, but flexes in the frontal plane. Flex sensors (not illustrated), are provided to output a signal indicative of the relative angle(s) between the upper strut 504-1 and the lower strut 504-2, and/or the central portion 502 and the upper strut 504-1 or the lower strut 504-2.

In the example shown in FIG. 5-2, the leg of the wearer (including upper leg 506-1 and lower leg 506-2) displays a valgus deformity in which the bone segment distal to the knee joint (i.e. the lower leg 506-2) is angled outward—i.e. angled laterally, away from the body's midline. The output of the flex sensor(s) enables a determination of the relative angle.

Conversely, in the example shown in FIG. 5-3 the leg of the wearer (including upper leg 506-1 and lower leg 506-2) displays a varus deformity in which the lower leg 506-2 is angled inward (i.e. angled laterally towards the body's midline). Again, the output of the flex sensor(s) enables a determination of the relative angle.

In examples, the wearable device may be configured to measure angulation in a single direction—for example the flex sensor may only exhibit a change in output due to angulation in a particular direction from a zero-reference position. In alternative examples, the wearable device may be configured to measure angulation in a both directions (e.g. inwards and outwards)—for example using a bi-directional flex sensor, or multiple flex sensors where one set is used for inward angulation and another set is used for outward angulation. In configurations where a bi-directional flex sensor is used, it is envisaged that a determination of the direction may be required using separate means to the flex sensor output alone—for example via user input or another sensor.

In an alternative example which is not illustrated, the wearable device may take the form of a sleeve of flexible material, with flex sensors positioned on one or more sides of the sleeve.

In another alternative example, which is not illustrated but will be described with reference to the knee brace 500, the upper strut 504-1 and lower strut 504-2 may be substantially rigid, but configured to pivot in the frontal plane relative to central portion 502. One or more angle sensors may be provided to measure the relative angle between the upper strut 504-1 and lower strut 504-2, to enable determination of the angulation of the leg.

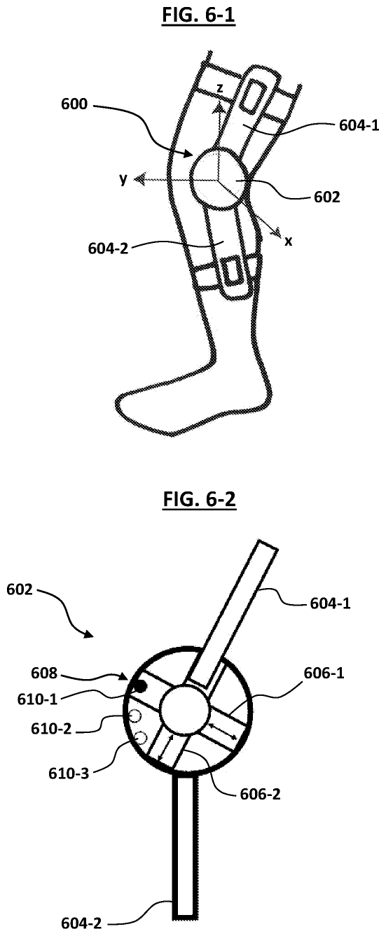

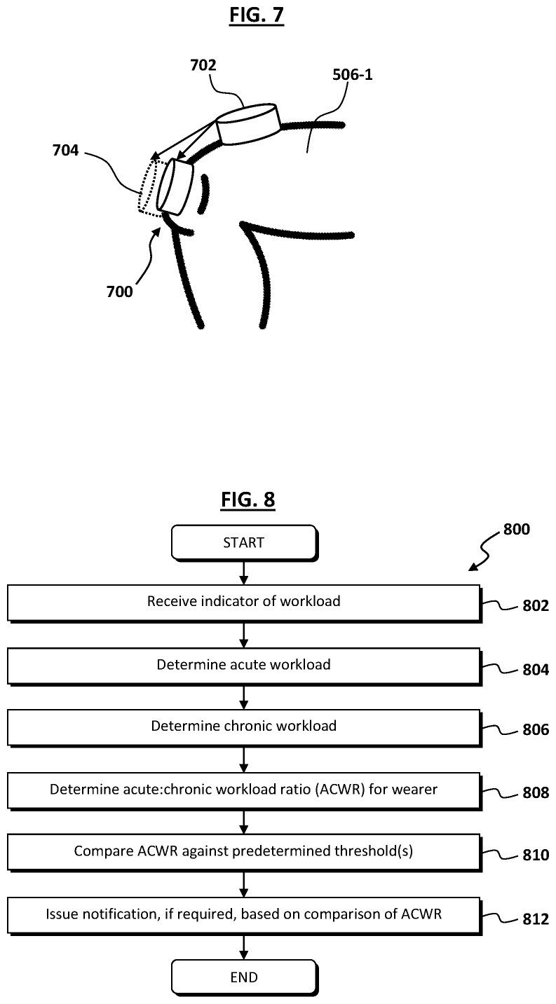

In certain forms of the present technology, a wearable device may be configured to measure at least one characteristic of a joint, or a body part associated with a joint, of a wearer. Referring to FIG. 6, a knee brace 600 includes a central joint assembly 602, a first body mounting portion in the form of upper strut 604-1, and a second body mounting portion in the form of lower strut 604-2.

In a first example, the joint assembly 602 is a planar mechanism configured to provide three degrees of freedom—for example along the x, y, z axes shown in FIG. 6—with the upper strut 604-1 and lower strut 604-2 locked in rotation to restrict movement between them to planar movement. Such an arrangement may provide a multi-axis or “xyz” stage with body attachment to each side of the stage.

Relative movement between the limb segments (i.e. the upper leg to which upper strut 604-1 is secured, and lower leg to which the lower strut 604-2 is secured) may be sensed using one or more sensors—for example, hall effect sensors and/or optical sensors. Joint laxity may be determined or inferred from this sensed movement.

In examples, the joint assembly 602 may be configured to selectively restrict movement to less than all degrees of freedom. For example, valgus rotation in the joint assembly 602 may be restricted (i.e. only sagittal plane motion possible), and the rotation locked through a pin mechanism so only relative planar movement of the knee can be sensed.

In a second example, the joint assembly 602 may include at least three linear joints having a three-axis orthogonal arrangement, each linear joint having an associated sensor configured to output an indication of movement in the associated linear joint (i.e. along a particular orthogonal axis).

Referring to FIG. 6-2, the joint assembly 602 illustrated includes a first slider 606-1 and a second slider 606-2, allowing movement in the x-y planes to measure laxity. Pin 608 may be selectively inserted into pin apertures 610-1 to 610-3 to lock rotation of the sliding joints at a desired angle.

In certain forms of the present technology, motion of a patella 700 of a person may be sensed. A reference sensor 702 is provided on the upper leg 506-1, while a motion parameter sensor unit 704 (for example, including an IMU) is provided on the patella 700. As the patella 700 is manipulated, movement data for motion of the sensor unit 704 relative to the reference sensor 702 is captured in order to enable assessment of characteristics such as laxity.

### 6.7 Determination of Workload and Monitoring Same

In certain forms of the present technology, a wearable device may be used to monitor workload of a wearer. In an example, the wearable device may be the first knee brace 200-1 or the second knee brace 200-2 as described above, in which knee angle travelled is monitored and used as an indicator of workload. It will be appreciated that the various processing steps described herein may be performed by one or more of at least: dedicated processor(s) of the knee brace 200, processor(s) of user devices 106, and/or the remote processing service 110.

FIG. 8 illustrates a method 800 of monitoring workload on a user—more particularly monitoring of an acute:chronic workload ratio (ACWR) for the wearer. In a first step 802, the angle of rotation of the knee to which the knee brace 200 is attached is received. In a second step 804, acute workload (for example, accumulated angle of rotation over a 24-hour period) for the wearer is determined. In a third step 806, chronic workload (for example, average daily accumulated angle of rotation over a one-week period) for the wearer is determined. In a fourth step 808, a current ACWR for the wearer is determined based on the acute workload and chronic workload.

In a fifth step 810, the current ACRW is compared with one or more predetermined thresholds for the user to assess a relative risk of injury or hampering recovery. For example, a first threshold of about 1 may be associated with a cautionary risk level, a second threshold of about 1.5 may be associated with a warning risk level, and a third threshold of about 2 may be associated with an alert risk level.

In examples, the predetermined thresholds may be time adjusted. Exemplary thresholds for an ACRW of “current day workload: past 7 day moving average workload” are provided in the table below:

| Time since surgery | Optimal ACWR | Cautionary | Warning | Alert |
|---|---|---|---|---|
| <1 month | 1.0-1.1 | 1.1-1.2 | 1.2-1.3 | >1.5 |
| 1-3 | 1.0-1.2 | 1.2-1.5 | 1.5-1.8 | >2.0 |
| 3-6 | 1.0-1.3 | 1.3-1.7 | 1.7-2.0 | >2.5 |
| 9-12 | 1.0-1.5 | 1.5-2.0 | 2.0-2.5 | >3.0 |

In a sixth step 812, if the current ACRW meets a predetermined threshold, at least one notification may be issued to the wearer, and/or a clinician or trainer of the wearer. In examples, the notification may be issued to a user device 106 (for example in a dedicated application, or as an electronic message). In examples, the notification may be issued at the knee brace 200—for example, a warning light, audible tone, or haptic feedback. In examples, the notification may include recommendations associated with the predetermined threshold—for example advice regarding managing the current workload experienced by the wearer.

Examples of notifications based on ACWR of “current day workload: past 7 day moving average workload” in the form of electronic messages are provided in the table below:

| ACWR | | | |
|---|---|---|---|
| threshold | | | |
| level | Timing | ACWR: Joint Angle | ACWR: Step count |
| Optimal | At end of day | “Good job, training | “Good job, you did the perfect |
| | | performed well today!” | number of steps today!” |
| Cautionary | At end of day | “You went a little hard | “You went a little hard today, |
| | | today, take it a bit easier | take a few less steps tomorrow” |
| | | tomorrow” | |
| Warning | As soon as | “You are pushing yourself | “You are doing too much |
| | ACWR hits this | too hard, please reduce | walking, please minimise steps |
| | threshold | exercise for the remainder | for the remainder of the day” |
| | | of the day” | |
| Alert | As soon as | “Alert, you are in danger of | “Alert, you are in danger of |
| | ACWR hits this | over exertion and are at | over exertion and are at risk of |
| | threshold | risk of doing damage!” | doing damage!” |

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Aspects of the present technology may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present technology.

1. A system for monitoring acute:chronic workload ratio (ACWR), the system including at least one processor configured to perform a method of:
receiving an indicator of workload from a device worn or carried by a user;
determining an acute workload for the user based on the received indicator of workload over a first period of time;
determining a chronic workload for the user based on the received indicator of workload over a second period of time, where the first period of time is shorter than the second period of time; and
determining a current ACWR for the user based on the acute workload and the chronic workload.

2. The system of claim 1, wherein the device is configured to measure an angle of rotation of a joint to which the device is mounted.

3. The system of claim 2, wherein the acute workload and the chronic workload are determined based on an accumulation of the angle of rotation of the joint over time.

4. The system of claim 1, wherein the device is configured to measure one or more of: distance travelled by the user, and one or more indicators of activity or posture by the user.

5. (canceled)

6. The system of claim 1, including estimating joint load of the user based on the received indicator of workload in conjunction with biomechanical modelling, and determining the acute workload and the chronic workload based on the estimated joint load.

7. The system of claim 1, wherein the chronic workload is determined as an average value of workload over the second period of time.

8. The system of claim 1, wherein the at least one processor is configured to determine a current risk level to the user based on the current ACWR.

9. The system of claim 8, wherein determining the current risk level includes comparing the current ACWR against one or more predetermined thresholds.

10. The system of claim 9, wherein the predetermined thresholds include a first threshold associated with a cautionary risk level, a second threshold associated with a warning risk level, and a third threshold associated with an alert risk level.

11. The system of claim 9, wherein the one or more predetermined thresholds are dynamic.

12. The system of claim 11, wherein the one or more predetermined thresholds increase based on time since an event.

13. The system of claim 11, wherein the one or more predetermined thresholds are adjusted based on performance metrics of the user.

14. The system of claim 1, wherein the at least one processor is configured to issue a notification based on the current risk level.

15. The system of claim 14, wherein the notification conveys information to the user regarding recommended activity based on the current risk level.

16. A computer-implemented method of monitoring acute:chronic workload ratio (ACWR), including:
receiving an indicator of workload from a device worn or carried by a user;
determining an acute workload for the user based on the received indicator of workload over a first period of time;
determining a chronic workload for the user based on the received indicator of workload over a second period of time, where the first period of time is shorter than the second period of time; and
determining a current ACWR for the user based on the acute workload and the chronic workload.

17. The computer-implemented method of claim 16, wherein the device is configured to measure an angle of rotation of a joint to which the device is mounted, and the acute workload and the chronic workload are determined based on an accumulation of the angle of rotation of the joint over time.

18. (canceled)

19. (canceled)

20. (canceled)

21. (canceled)

22. (canceled)

23. The computer-implemented method of claims 16, including determining a current risk level to the user based on the current ACWR.

24. The computer-implemented method of claim 23, wherein determining the current risk level includes comparing the current ACWR against one or more predetermined thresholds.

25. (canceled)

26. The computer-implemented method of claim 24, wherein the one or more predetermined thresholds are dynamic.

27. (canceled)

28. (canceled)

29. The computer-implemented method of claim 23, including issuing a notification based on the current risk level.

30. (canceled)
